(19) United States
(12) Patent Application Publication
(10) Pub. No.: US 2026/0077154 A1
(43) Pub. Date: Mar. 19, 2026

(54) Wearable Electronic Device

(71) Applicants: Michael Huff, Pasadena, CA (US); Alexis Alexander, Austin, TX (US); Austin Huff, Arcadia, CA (US); Jonathan Hall, Irvine, CA (US)

(72) Inventor: Michael Huff, Pasadena, CA (US)

(21) Appl. No.: 19/318,726

(22) Filed: Sep. 4, 2025

Related U.S. Application Data
(60) Provisional application No. 63/695,300, filed on Sep. 16, 2024.

(51) Int. Cl. A61M 21/02; A61M 21/00

(52) U.S. Cl. CPC A61M 21/02; A61M 2021/0088; A61M 2205/18; A61M 2205/3303; A61M 2205/502; A61M 2209/088

(57) ABSTRACT

A wearable device, method and system for assisting a user in mitigating an amygdala hijack event is provided. The method includes: receiving physiological data of the user from one or more sensors; analyzing the physiological data to determine whether the data is indicative of the user experiencing an amygdala hijack event; and upon determining that the data is indicative of the user experiencing an amygdala hijack event, initiating an intervention on an output interface to mitigate the amygdala hijack event.

### CROSS-REFERENCE TO RELATED APPLICATION

This application claims priority to U.S. Provisional Patent Application 63/695,300, filed Sep. 16, 2024, which is incorporated herein by reference.

### TECHNICAL FIELD

The present invention relates to wearable electronic devices. In particular, but not exclusively, the present invention relates to wearable devices for monitoring physiological characteristics of a user.

### BACKGROUND

Amygdala hijacking is a term that describes an immediate and overwhelming emotional response that is often disproportionate to the circumstances. In short, the emotional part of the brain overpowers the rational part, often leading to impulsive and irrational behavior. This impulsive and irrational behavior may have a variety of negative impacts, including in relation to personal safety and wellbeing.

Several techniques exist that are able to reduce the occurrence of amygdala hijacking, including mindfulness techniques or cognitive behavioral therapy (CBT). These techniques are, however, not immediately effective, and generally require significant and active participation from the individual.

Furthermore, several techniques exist that are able to help individuals to stop amygdala hijacking when it starts. These often rely on teaching the person to identify an amygdala hijack, such as emotional and physical symptoms, as well as teaching specific techniques to the user to stop the amygdala hijack.

A problem with such techniques is that it is generally very difficult for people to identify an amygdala hijack as it is happening. As a result, it requires significant effort from an individual to be able to learn to identify an amygdala hijack.

As such, there is clearly a need for improved techniques to address amygdala hijacking.

It will be clearly understood that, if a prior art publication is referred to herein, this reference does not constitute an admission that the publication forms part of the common general knowledge in the art in Australia or in any other country.

### SUMMARY OF INVENTION

Embodiments of the present invention provide wearable electronic devices, which may at least partially address one or more of the problems or deficiencies mentioned above or which may provide the public with a useful or commercial choice.

With the foregoing in view, according to a first aspect, the invention resides broadly in a wearable electronic device adapted to be worn by a user, comprising:
a processor;
one or more sensors, coupled to the processor;
an output interface, coupled to the processor; and
a memory, coupled to the processor, storing executable instructions that, when executed by the processor, cause the processor to:
   receive physiological data from the one or more sensors;
   analyze the physiological data to determine whether the data is indicative of the user experiencing an amygdala hijack event;
   upon determining that the data is indicative of the user experiencing an amygdala hijack event, initiate an intervention on the output interface to mitigate the amygdala hijack event.

Advantageously, the wearable electronic device is able to identify data that is indicative of the user experiencing an amygdala hijack event, and to initiate an intervention on the output interface to mitigate the amygdala hijack event. This may in turn restore parasympathetic nervous system function, and avoid or reduce impulsive and irrational behavior associated with the amygdala hijack event. The wearable device may also enable a user to mitigate amygdala hijack events without requiring any significant skill of the user.

The wearable electronic device may comprise a smartwatch. The wearable electronic device may comprise smart glasses. The wearable electronic device may comprise a smart ring, or any other suitable wearable electronic device.

The wearable electronic device may be rechargeable. In particular, the wearable device may include a rechargeable battery configured to power the wearable electronic device.

Preferably, the sensors are non-invasive sensors. The sensors may be skin contact sensors.

Preferably, the sensors include electrodermal activity (EDA) sensors.

The sensors may include one or more of galvanic skin response (GSR) sensors, skin conductance (SC) sensors, electrodermal response (EDR) sensors, and psychogalvanic reflex (PGR) sensors.

The sensors may include a pulse oximeter. The pulse oximeter may be used to determine an oxygen saturation level of the user.

The sensors may include a heart rate sensor. The heart rate sensor may be configured to determine a heart rate of the user. The sensors may include an optical heart rate (OHR) sensor.

The sensors may include a skin-temperature sensor, configured to determine a skin temperature of the user.

The sensors may include a blood pressure sensor. The blood pressure sensor may comprise a cuffless photoplethysmography sensor.

The sensors may include an accelerometer. The sensors may include a gyroscope. The sensors may include an inertial measurement unit (IMU).

The sensors may comprise a plurality of sensors. The plurality of sensors may comprise different types of sensors.

The output interface may comprise a display screen. The display screen may be configured to display instructions to the user.

The intervention may comprise a breathing intervention. In such case, the instructions may comprise guided breathing instructions.

The display screen may comprise a holographic display screen.

The output interface may comprise a speaker. The speaker may be configured to issue an audible alarm, e.g. to attract the attention of the user. The speaker may be configured to issue verbal instructions to the user.

The output interface may comprise a haptic actuator. The haptic actuator may be configured to vibrate to attract the attention of the user.

The output interface may comprise a light. The light may be configured to flash to attract the attention of the user.

The output interface may comprise an olfactory output interface. The olfactory output interface may comprise an atomizer or thermal evaporator.

The output interface may be one of a plurality of output interfaces, wherein the intervention is initiated on the plurality of output interfaces. For example, the intervention may utilize each of the plurality of output interfaces.

The intervention may be of any suitable form. Preferably the intervention is a behavioral psychology-based intervention.

The intervention may include an output on the output interface, such as an alert, to attract the attention of the user.

The intervention may be provided using voice coaching. The voice coaching may be provided on a speaker of the wearable device.

The intervention may be provided using textual coaching. The textual coaching may be provided on a display of the wearable device.

The intervention may include presenting a grounding routine to the user. The grounding routine may, for example, prompt the user to identify five visual, four tactile, three auditory, two olfactory, and one gustatory stimulus.

The intervention may include guided breathing. The intervention may include audible or visual instructions guiding the user in guided breathing.

The intervention may include a user input component. The user input component may include pressing a button on the wearable electronic device.

The intervention may include cognitive reappraisal prompts. The cognitive reappraisal prompts may include an “Identify the thought” prompt, prompting the user to identify and name their thought(s), a “Rate evidence” prompt, prompting the user to rate evidence to support the thought, and an “Alternative view” prompt, prompting the user to present an alternative view.

The intervention may include tapping sequences illustrated on a display screen with timing cues.

The intervention may dynamically change based upon sensor data. For example, a guided breathing intervention may dynamically change based on heart rate data. The breathing intervention may stop when the sensor data, or a portion thereof, goes below a threshold.

The intervention may continue until parasympathetic restoration is determined based on the sensor data.

The wearable electronic device may prompt the user to acknowledge the intervention when the processor determines that the data is indicative of the user experiencing an amygdala hijack event, and issue an alert if no user acknowledgement is provided within a predefined period. The predefined period may be determined from when the processor determining that the data is indicative of the user experiencing an amygdala hijack event. The alert may comprise an audible, visual and/or haptic alert.

The analysis of the physiological data may include comparing the physiological data to one or more thresholds. The thresholds may be static or dynamic.

The thresholds may be user-specific thresholds. The thresholds may be determined based on historical data.

The analysis of the physiological data may include a time-based analysis. For example, the physiological data may be analyzed for sudden changes.

The analysis of the physiological data may include trend-based analysis. For example, a galvanic skin response (GSR) slope may be determined and compared to a threshold.

The analysis of the physiological data may include artificial intelligence (AI) analysis. The AI analysis may be performed using a model on the wearable electronic device.

The analysis of the physiological data may be performed using a convolutional neural network.

The analysis of the physiological data may be performed using a recurrent neural network.

According to a second aspect, the present invention resides broadly in a system for assisting a user in mitigating an amygdala hijack event, the system including a wearable device according to the first aspect.

The system may further include a computing device, coupled to the wearable device, for analyzing the sensor data.

The computing device may be a remote computing device. The computing device may comprise an AI computing device.

The computing device may include a portable computing device. The portable computing device may comprise a smartphone.

According to a third aspect, the present invention resides broadly in a method for assisting a user in mitigating an amygdala hijack event, the method including:
receiving physiological data of the user from one or more sensors;
analyzing the physiological data to determine whether the data is indicative of the user experiencing an amygdala hijack event;
upon determining that the data is indicative of the user experiencing an amygdala hijack event, initiating an intervention on an output interface to mitigate the amygdala hijack event.

The method may be performed on a wearable device according to the first aspect.

According to a fourth aspect, the present invention resides broadly in a system for assisting a user in mitigating an amygdala hijack event, the system including:
a processor;
one or more wearable sensors, coupled to the processor;
an output interface, coupled to the processor; and
a memory, coupled to the processor, storing executable instructions that, when executed by the processor, cause the processor to:
   receive physiological data from the one or more wearable sensors;
   analyze the physiological data to determine whether the data is indicative of the user experiencing an amygdala hijack event;
   upon determining that the data is indicative of the user experiencing an amygdala hijack event, initiate an intervention on the output interface to mitigate the amygdala hijack event.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 illustrates a simplified diagram of a system for assisting a user in mitigating an amygdala hijack event, according to an embodiment of the present invention.

FIG. 2 illustrates a front view of the smartwatch including a first intervention screen displayed thereon, according to an embodiment of the present invention.

FIG. 3 illustrates a front view of the smartwatch including a second intervention screen displayed thereon, according to an embodiment of the present invention.

FIG. 4 illustrates a front view of the smartwatch including a third intervention screen displayed thereon, according to an embodiment of the present invention.

FIG. 5 illustrates a schematic of a wearable device, according to an embodiment of the present invention.

FIG. 6 illustrates a method for assisting a user in mitigating an amygdala hijack event, according to an embodiment of the present invention.

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

### DETAILED DESCRIPTION

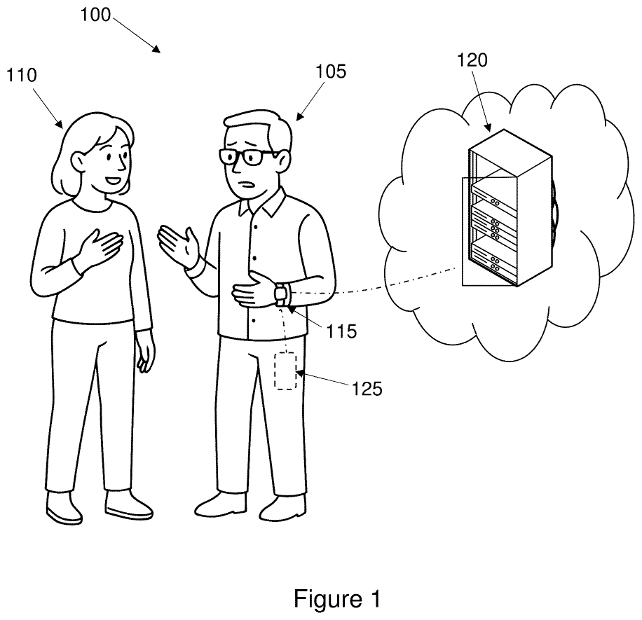

FIG. 1 illustrates a simplified diagram of a system 100 for assisting a user 105 in mitigating an amygdala hijack event, according to an embodiment of the present invention.

The system 100 is illustrated with reference to a stressful environment where the user 105 is in discussion with another person 110. The skilled addressee will, however, readily appreciate that the system 100 may be used in any suitable environment.

The system 100 includes a wearable electronic device in the form of a smartwatch 115, worn by the user 105.

The smartwatch 115 includes sensors, configured to capture physiological data of the user 105, and a processor coupled to the sensors, configured to analyze the physiological data to determine whether the data is indicative of the user 105 experiencing an amygdala hijack event.

The smartwatch 115 further includes output interfaces, coupled to the processor. When the physiological data is indicative of the user 105 experiencing an amygdala hijack event, an intervention is initiated on the output interfaces to mitigate the amygdala hijack event.

The one or more sensors are non-invasive sensors, and include a galvanic skin response (GSR) sensor, and a heart rate sensor. The skilled addressee will, however, readily appreciate that any suitable type of sensor may be used, including electrodermal activity (EDA) sensors, skin conductance (SC) sensors, electrodermal response (EDR) sensors, psychogalvanic reflex (PGR) sensors, pulse oximeters, skin-temperature sensors, accelerometers, gyroscopes, and an inertial measurement unit (IMU).

The data from the galvanic skin response (GSR) sensor, and the heart rate sensor are analyzed using any suitable algorithm. In some embodiments, the data is analyzed using Artificial Intelligence (AI) or Machine Learning (ML), as outlined below.

The output interface comprises a haptic output interface, a speaker, and a touchscreen display. When the physiological data is indicative of the user 105 experiencing an amygdala hijack event, the haptic output interface initially vibrates, drawing the attention of the user 105 away from the situation they are in, and to the smartwatch 115. The speaker also outputs an alarm, to also draw the attention of the user 105 away from the situation they are in, and to the smartwatch 115. Finally, the touchscreen display is configured to display instructions to the user 105.

The display instructions may take any suitable form, and preferably comprise a behavioral psychology-based intervention. This could include various types of coaching, breathing instructions, a grounding routine, or the like.

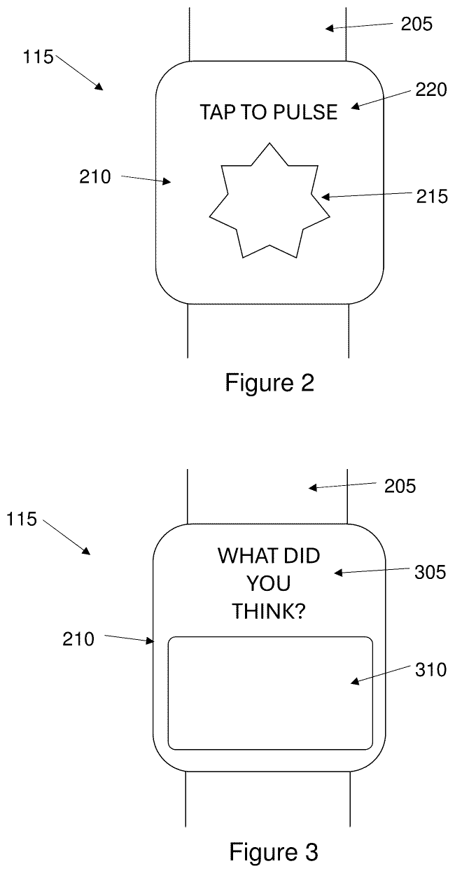

FIG. 2 illustrates a front view of the smartwatch 115 including a first intervention screen displayed thereon, according to an embodiment of the present invention.

The smartwatch 115 includes a strap 205, for attaching to a wrist of the user, and a touchscreen display 210.

In FIG. 2, an intervention in the form of tapping sequences is illustrated on the touchscreen display 210 with timing cues. In particular, the display includes a star element 215, which pulses, wherein the user 105 is instructed to tap the touchscreen display 210 at a rate of the pulses with an instruction prompt 220.

The content on the touchscreen display 210 is dynamically updated based on interaction of the user 105 therewith. For example, the star element 215 may change color when the user 105 taps the touchscreen display 210.

The tapping exercise is designed to remove the focus of the user 105 from the stressful situation, to the smartwatch 115, to ground the user 105.

While this intervention is tapping based, in other embodiments, the intervention may include other types of interventions. Furthermore, the intervention may be configurable by the user 105 based on what works for them.

The intervention may further include multiple stages, with different purposes. The multiple stages may include an initial distraction stage, and a cognitive reappraisal stage, for example. The cognitive reappraisal stage may include reappraisal prompts, e.g. an “Identify the thought” prompt, prompting the user to identify and name their thought(s), a “Rate evidence” prompt, prompting the user to rate evidence to support the thought, and an “Alternative view” prompt, prompting the user to present an alternative view.

FIG. 3 illustrates a front view of the smartwatch 115 including a second intervention screen displayed thereon, according to an embodiment of the present invention.

The second intervention screen includes an “Identify the thought” prompt 305, prompting the user to identify and name their thought(s), and a text box 310, in which the user is able to write down their thoughts.

Prompting the user to identify and name their thoughts generally assists the user in challenging unhelpful and irrational negative thoughts and beliefs, e.g. in a cognitive behavioral therapy (CBT) type manner.

The intervention may finish with a review stage, where the user is prompted to provide feedback.

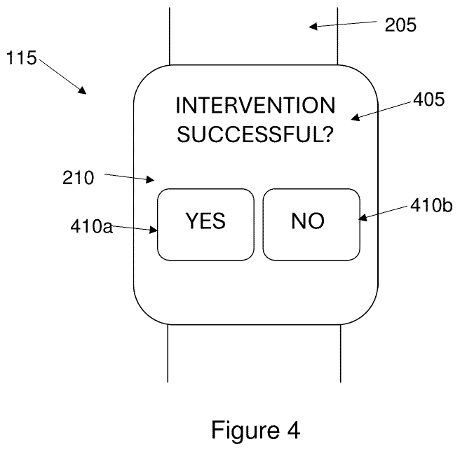

FIG. 4 illustrates a front view of the smartwatch 115 including a third intervention screen displayed thereon, according to an embodiment of the present invention.

The third intervention screen includes a prompt 405, prompting the user to respond with whether the intervention was successful, and includes “yes” and “no” buttons 410a, 410b, to enable the user to respond to the prompt.

Upon receipt of the user's answer, the answer is provided to an Artificial Intelligence (AI) or Machine Learning (ML) model to enable the system 100 to learn based on feedback.

In particular, the AI or ML model is able to use actual feedback from the user, together with physiological data, to determine which interventions work best for which users in which circumstances.

The AI or ML model is then able to propose interventions in case of future amygdala hijack events based on such determination.

As an illustrative example, the physiological data of the user and the outcome of the intervention are provided to the AI or ML model as they become available. It is envisaged that many users use smartwatches 115, and as such, data from many users is provided to the AI or ML model.

Physiological data from the smartwatch 115 is periodically provided to the AI or ML model, initially to determine whether the user is likely to be in or near an amygdala hijack event.

The AI or ML model may be a prompt-based model, and in such case, the smartwatch 115 may generate a prompt for the AI or ML model asking a likelihood of the user being in or near an amygdala hijack event.

When it is determined that the user is in or near an amygdala hijack event (e.g. a risk or likelihood is above a threshold), then the AI or ML model may select an intervention of a plurality of possible interventions.

Now turning back to FIG. 1, the system 100 includes an external computing device in the form of a server 120, with which the smartwatch 115 is connected.

While the server 120 is illustrated as a single device, the skilled addressee will readily appreciate that the server 120 may comprise a plurality of devices operating in a distributed manner, as is common in the case of cloud computing.

The server 120 includes an AI model, and is configured to receive review data based on the success of interventions, and propose future interventions based thereon.

The AI model may include a convolutional neural network, a recurrent neural network, or any suitable type of network.

The smartwatch 115 may further submit physiological data of the user 105 to the server 120, wherein the AI model is configured to propose interventions at least in part based on the physiological data. As outlined above, the AI model may include or be trained by physiological data and/or intervention success data of a plurality of users. The AI model may further use user preferences and other specific data of the user 105 when proposing an intervention.

While in FIG. 1 the server 120 and AI model are illustrated as being external to the smartwatch, in other embodiments, the AI model (or ML model) is provided in the smartwatch 115 itself.

The smartwatch 115 is further coupled to a smartphone 125 of the user 105. The smartphone 125 may work with the smartwatch 115 to process the data and/or to identify an amygdala hijack event. Furthermore, at least part of the intervention may be performed on the smartphone 125.

While the above embodiment relates to a smartwatch 115, in another embodiment, the wearable electronic device may comprise a smart ring, smart glasses, or any other suitable wearable device.

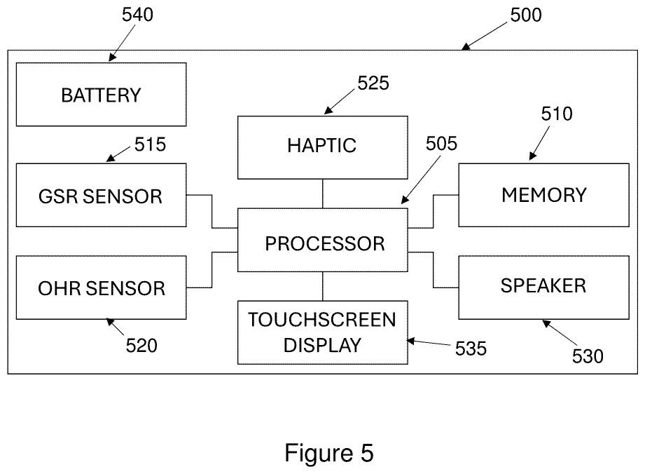

FIG. 5 illustrates a schematic of a wearable device 500, according to an embodiment of the present invention. The wearable device 500 may be similar or identical to the smartwatch 115.

The wearable device 500 includes a processor 505, and a memory 510 coupled to the processor 505. The memory 510 includes instruction code, executable by the processor 505, for performing various functions of the wearable device 500.

The wearable device 500 further includes a plurality of sensors in the form of a Galvanic Skin Response (GSR) sensor 515 and an Optical Heart Rate (OHR) sensor 520, and a plurality of output interfaces in the form of a haptic output interface 525, a speaker 530 and a touchscreen display 535.

The GSR sensor 515 and the OHR sensor 520 capture physiological data of the user, which is provided to the processor 505, which determines whether the user is experiencing an amygdala hijack event. If the user is experiencing an amygdala hijack event, an intervention is provided on the haptic output interface 525, speaker 530 and touchscreen display 535. The intervention may be similar or identical to that provided above.

The wearable device 500 includes a rechargeable battery 540 and is thus rechargeable. The rechargeable battery 540 powers the various elements of the wearable device 500. While not illustrated, the wearable device 500 may include a charging interface (e.g., wireless charging or USB-C port).

As outlined above, the intervention may take a number of forms. In some embodiments, the intervention may dynamically change based upon sensor data, e.g. data from the GSR sensor 515 and the OHR sensor 520.

As an illustrative example, the intervention may comprise a guided breathing intervention, where the user is guided to breathe according to instructions on the touchscreen display 535. The instructions on the display may dynamically change based on heart rate data from the OHR sensor 520, e.g. by changing a rate in which the breathing is performed, or ending the intervention when the heart rate drops below a certain level.

The wearable device 500 may further request that the user acknowledges the intervention, e.g. through interaction with the touchscreen display 535.

The wearable device 500 may issue an alert if no user acknowledgement is provided within a predefined period. The alert may comprise an audible, visual and/or haptic alert. Alternatively or additionally, the alert may be in the form of a message or notification to the smartphone 125 or a third-party device.

As outlined above, the analysis of the physiological data may be performed in any suitable way, including using AI and ML. In other embodiments, the physiological data is processed and compared to one or more thresholds, that may be static or dynamic. In some embodiments, the thresholds are user specific thresholds, and may be determined based on historical data.

For example, if a user's physiological data is typically within a certain range, the thresholds may be set such that they are outside of that range.

Furthermore, the analysis of the physiological data may include a time-based analysis. For example, the physiological data may be analyzed for sudden changes.

The analysis of the physiological data may be performed using a convolutional neural network or a recurrent neural network, or any other form of AI or ML model.

While not illustrated, the wearable device may include a data interface, such as a radio interface (e.g., Bluetooth, Wi-Fi, or NFC) for provisioning and data synchronization.

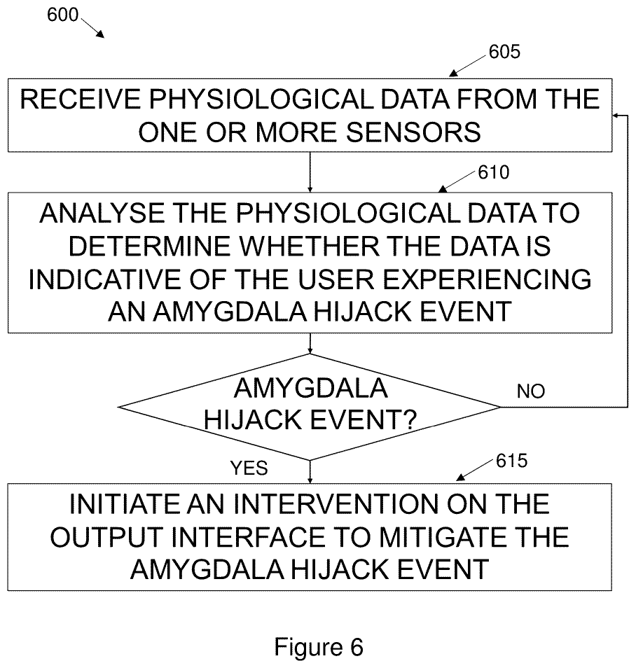

FIG. 6 illustrates a method 600 for assisting a user in mitigating an amygdala hijack event, according to an embodiment of the present invention. The method 600 may be similar or identical to the method performed by the smartwatch 115 or wearable device 500.

At step 605, physiological data of the user is received from one or more sensors, such as a GSR sensor 515 and/or an OHR sensor. The sensors may be sensors of a wearable device, such as the wearable device 500 or smartwatch 115.

At step 610, the physiological data is analyzed to determine whether the data is indicative of the user experiencing an amygdala hijack event. The analysis may be performed in any suitable way, including with use of AI.

If the data is indicative of the user experiencing an amygdala hijack event, an intervention is initiated on an output interface, such as a display, or similar, to mitigate the amygdala hijack event at step 615.

Otherwise, the process repeats at step 605, to ensure that the physiological data is constantly monitored and analyzed.

The system may include an application on the smartphone or other device, or provide a web platform that presents selected interventions and tracks progress with individualized adaptation. For example, the system may plot on an application or web platform a rate of amygdala hijack events, intervention success rate, or any suitable data.

While the above systems illustrate the wearable devices 100, 500 communicating with external devices, the skilled addressee will readily appreciate that the wearable devices 100, 500 may be standalone devices. In such case, the wearable devices 100, 500 may perform all functionality on the device itself, rather than needing to send sensitive data externally. Furthermore, any sensitive data stored on the device may be encrypted. One or more of these features may reduce the likelihood that personal data is compromised.

Advantageously, the systems, methods and devices outlined above are able to identify data that is indicative of the user experiencing an amygdala hijack event, and to initiate an intervention on the output interface to mitigate the amygdala hijack event. This may in turn restore parasympathetic nervous system function, and avoid or reduce impulsive and irrational behavior associated with the amygdala hijack event. The systems, methods and devices may also enable a user to mitigate amygdala hijack events without requiring any significant skill of the user, and as such, are accessible.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A wearable electronic device adapted to be worn by a user, comprising:
a processor;
one or more sensors, coupled to the processor;
an output interface, coupled to the processor; and
a memory, coupled to the processor, storing executable instructions that, when executed by the processor, cause the processor to:
receive physiological data from the one or more sensors;
analyze the physiological data to determine whether the physiological data is indicative of the user experiencing an amygdala hijack event; and
upon determining that the physiological data is indicative of the user experiencing the amygdala hijack event, initiate an intervention on the output interface to mitigate the amygdala hijack event.

2. The wearable electronic device of claim 1, wherein the wearable electronic device comprises a smartwatch, smart glasses, or a smart ring.

3. The wearable electronic device of claim 1, wherein the one or more sensors are non-invasive sensors.

4. The wearable electronic device of claim 1, wherein the one or more sensors comprise electrodermal activity (EDA) sensors.

5. The wearable electronic device of claim 1, wherein the one or more sensors comprise one or more of galvanic skin response (GSR) sensors, skin conductance (SC) sensors, electrodermal response (EDR) sensors, or psychogalvanic reflex (PGR) sensors.

6. The wearable electronic device of claim 1, wherein the one or more sensors comprise a pulse oximeter, to determine an oxygen saturation level of the user.

7. The wearable electronic device of claim 1, wherein the one or more sensors comprise a heart rate sensor, configured to determine a heart rate of the user.

8. The wearable electronic device of claim 1, wherein the one or more sensors comprise a skin temperature sensor, configured to determine a skin temperature of the user.

9. The wearable electronic device of claim 1, wherein the one or more sensors comprise at least one of an accelerometer, a gyroscope, or an inertial measurement unit (IMU).

10. The wearable electronic device of claim 1, wherein the one or more sensors comprise a plurality of sensors, and wherein the plurality of sensors comprise different types of sensors.

11. The wearable electronic device of claim 1, wherein the output interface comprises a display screen, configured to display instructions to the user.

12. The wearable electronic device of claim 11, wherein the intervention comprises a breathing intervention, wherein the instructions comprise guided breathing instructions.

13. The wearable electronic device of claim 1, wherein the output interface comprises a speaker, configured to at least one of: 1) issue an audible alarm to attract attention of the user, or 2) issue verbal instructions to the user.

14. The wearable electronic device of claim 1, wherein the output interface comprises a haptic actuator, configured to vibrate to attract attention of the user.

15. The wearable electronic device of claim 1, wherein the output interface is one of a plurality of output interfaces, wherein the intervention is initiated on the plurality of output interfaces.

16. The wearable electronic device of claim 1, wherein the intervention dynamically changes based upon sensor data.

17. The wearable electronic device of claim 1, wherein the analysis of the physiological data comprises comparing the physiological data to one or more thresholds.

18. The wearable electronic device of claim 1, wherein the analysis of the physiological data comprises artificial intelligence (AI) analysis, performed using an AI model on the wearable electronic device.

19. A system for assisting a user in mitigating an amygdala hijack event, the system comprising:
a processor;
one or more wearable sensors, coupled to the processor;
an output interface, coupled to the processor; and
a memory, coupled to the processor, storing executable instructions that, when executed by the processor, cause the processor to:
receive physiological data from the one or more wearable sensors;
analyze the physiological data to determine whether the physiological data is indicative of the user experiencing the amygdala hijack event; and
upon determining that the physiological data is indicative of the user experiencing the amygdala hijack event, initiate an intervention on the output interface to mitigate the amygdala hijack event.

20. A method for assisting a user in mitigating an amygdala hijack event, the method comprising:
receiving physiological data of the user from one or more sensors;
analyzing the physiological data to determine whether the physiological data is indicative of the user experiencing the amygdala hijack event; and
upon determining that the physiological data is indicative of the user experiencing the amygdala hijack event, initiating an intervention on an output interface to mitigate the amygdala hijack event.
